(19) United States
(12) Patent Application Publication
(10) Pub. No.: US 2008/0033227 A1
(43) Pub. Date: Feb. 7, 2008

(54) Recovery of sulfur value in an alkylation process

(76) Inventor: David Campbell Graves, Kansas City, MO (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/890,054

(22) Filed: Aug. 3, 2007

Related U.S. Application Data
(60) Provisional application No. 60/835,390, filed on Aug. 3, 2006.

(51) Int. Cl. C07C 6/08

(52) U.S. Cl. 585/707; 585/708

(57) ABSTRACT

The present invention provides an improved sulfuric acid catalyzed alkylation process wherein the sulfur content from one or more streams in the alkylation process is recovered by an extraction agent. The extraction agent of the invention comprises ammonia or ammonium hydroxide. Advantageously, the recovered sulfur content is converted to sulfuric acid, such as in a spent acid regeneration (SAR) plant, and recycled to replenish decomposed catalyst in the alkylation process.

### FIELD OF THE INVENTION

The present invention relates to a sulfuric acid catalyzed alkylation process and in particular, to an alkylation process wherein the sulfur content from sulfur contaminated streams in the alkylation process is recovered by an extraction agent.

### BACKGROUND OF THE INVENTION

In a typical sulfuric acid catalyzed alkylation process, light olefins (e.g., C₃-C₅ olefins) are contacted with a low molecular weight isoparaffin hydrocarbon (e.g., isobutane) in the presence of sulfuric acid (as a strong acid catalyst) in an alkylation reactor under conditions causing the protonation and conversion of the olefins into carbocations which in turn react with isobutane to produce a higher molecular weight alkylate product. This alkylate product is typically used as blending stock to obtain higher octane gasoline; e.g., gasoline having motor octane numbers from about 88 to 95 and research octane numbers from about 90 to 98. Alkylation chemistry is generally described by Kranz in “Alkylation Chemistry, Mechanisms, Operating Variable and Olefin Interactions”, available at http://www.stratcoalkylation.com, accessed Jul. 21, 2006, hereby incorporated by reference. Branzaru generally describes an alkylation unit process design in “Introduction to Sulfuric Acid Alkylation Unit Process Design”, also available at http://www.stratcoalkylation.com, accessed Jul. 21, 2006, hereby incorporated by reference.

Two important requirements of typical sulfuric acid catalyzed alkylation process are: 1) the replenishment of sulfuric acid catalyst, and 2) the removal of sulfur contaminants from various process streams. Catalyst replenishment is required, inter alia, because of the decomposition of the sulfuric acid catalyst and to prevent the concomitant lowered production yield. Sulfur contaminants are, inter alia, derived from the sulfuric acid catalyst and include: 1) sulfur dioxide, derived from the decomposition of sulfuric acid catalyst; and 2) alkyl and dialkyl sulfates, derived from reactions of sulfuric acid with hydrocarbons. The term “sulfur contaminant” herein includes any derivative of sulfuric acid made during an alkylation process and also includes sulfuric acid no longer being used as a catalyst.

Instead of converting sulfur contaminants into useful compounds (such as sulfuric acid for catalyst replenishment), the sulfur value of these sulfur contaminants is typically lost and discarded in alkylation process waste streams. Sulfur contaminants are removed in a typical alkylation process by contacting a hydrocarbon containing liquid or gas stream with aqueous caustic (sodium hydroxide) thereby extracting the sulfur contaminants (as their corresponding sodium salts) into the aqueous phase and resulting in aqueous waste streams. These waste streams, also known as “waste caustic” or spent “alkaline water,” are discarded and represent a significant loss of sulfur value.

For example, alkyl sulfate is typically separated from the alkylate product by first adding concentrated sulfuric acid to dissolve some of the alkyl sulfates followed by washing with aqueous caustic thereby neutralizing any carry over of strong acid, and hydrolyzing and dissolving any residual acidic species. Sulfur dioxide by-product and hydrocarbon gases are typically liberated from the alkylate product into a vapor phase known as “refrigerant”. A portion of the refrigerant is typically used as a coolant while another portion, called the “depropanizer feed stream,” is typically sent to a distillation tower to remove propane. Prior to the distillation tower, the depropanizer feed stream is usually treated with aqueous caustic, inter alia, to extract the sulfur dioxide into an aqueous solution.

Typically, a high concentration of aqueous caustic is avoided because of its corrosive effects on process equipment. Unfortunately, lower concentrations of caustic necessary to avoid corrosion result in decreased removal of sulfur contaminants from hydrocarbon streams and thereby generate waste caustic having an undesirably high dilution of sulfur. While it would be advantageous to injected such waste into a spent acid regeneration furnace, energy demands would become cost prohibitive to maintain the furnace temperature required for incineration of such high aqueous dilutions of sulfur. Furthermore, incinerating waste caustic would undesirably form large amounts of sodium containing ash. Thus, in conventional practice, it is more economical and efficient to dispose of waste caustic in waste treatment and to add make-up sulfur to a spent acid regeneration plant rather than attempt to recover sulfur value from waste caustic.

It would therefore be desirable to discover an alternative to the use of aqueous caustic to avoid the aforementioned disadvantages. Müller et al., in Ullmann's Encyclopedia of Industrial Chemistry, Wiley-VCH Verlag GmbH & Co. KGaA (Article Online Posting Date: Jun. 15, 2000, downloaded 12 Dec. 2005) in “Ammonia Washing”, disclose use of an ammonium hydroxide solution to scrub a gas stream containing sulfur dioxide, sulfur trioxide, and sulfuric acid to produce an ammonium salt solution. Müller et al. describe disposing of this ammonium salt solution, predominately a sulfite solution, by incineration in a furnace, wherein the ammonium salts are converted to elemental nitrogen and sulfur oxides. Unfortunately, Müller et al. do not show how to extract sulfur contaminants from an alkylation stream.

### SUMMARY OF THE INVENTION

A sulfuric acid catalyzed alkylation process has now been discovered which provides for the recovery of sulfur contaminants from hydrocarbon streams and conversion thereof into sulfuric acid for catalyst replenishment. The alkylation process of the invention eliminates or reduces the use of aqueous caustic for removal of sulfur contaminants from hydrocarbon streams. The invention also eliminates or reduces the generation of non-recycled waste caustic.

The present invention provides an improved sulfuric acid alkylation process wherein the sulfur content formerly discarded in conventional process waste streams is recovered and used to re-form sulfuric acid, which can then be recycled to the alkylation process.

In a broad sense, the invention uses ammonia or ammonium hydroxide to extract sulfur contaminants from any alkylation process stream having hydrocarbons.

In one embodiment, the alkylation process provided by the invention comprises the steps of:
a) generating at least one sulfur containing stream by reacting olefins and isobutane in the presence of a sulfuric acid catalyst in an alkylation reactor to produce said sulfur containing stream which comprises hydrocarbons and sulfur contaminants;
b) extracting an amount of the sulfur contaminants from said sulfur containing stream by contacting said sulfur containing stream with ammonia or ammonium hydroxide to produce a treated stream and an ammonium salt solution comprising an extracted amount of an ammonium salt derived from said sulfur contaminants; and
c) separating said ammonium salt solution from the treated stream.

The alkylation process provided by the invention can further comprise the steps of:
d) incinerating said ammonium salt solution in a furnace, preferably a spent acid regeneration furnace, to produce a gaseous mixture comprising N₂, NO_{X}, SO₂, SO₃;
e) contacting said gaseous mixture with oxygen in the presence of an oxidation catalyst, preferably vanadium oxide, to convert SO₂ to SO₃; and
f) contacting said gaseous mixture with water to form sulfuric acid from the SO₃ therein.

The alkylation process provided by the invention can further comprise the step of replenishing said sulfuric acid catalyst in step a) with the sulfuric acid made in step f).

Suitably, at least one sulfur containing stream in step a) is a liquid made by a method comprising the steps of:
i) reacting said olefins and said isobutane in the presence of said sulfuric acid catalyst in said alkylation reactor to produce an alkylate stream and a spent acid stream;
ii) separating the alkylate stream into a vapor stream and a liquid effluent stream; and
iii) compressing and condensing the vapor stream thereby forming the sulfur containing stream.

Also suitably, at least one sulfur containing stream in step a) is a liquid made by a method comprising the steps of:
i) reacting said olefins and said isobutane in the presence of said sulfuric acid catalyst in said alkylation reactor to produce an alkylate stream and a spent acid stream;
ii) separating the alkylate stream into a vapor stream and a liquid effluent stream;
iii) optionally contacting the liquid effluent stream with an acid; and
iv) obtaining the sulfur containing stream from the liquid component.

Also suitably, at least one sulfur containing stream in step a) is a gas made by a method comprising the steps of:
i) reacting olefins and isobutane in the presence of said sulfuric acid catalyst in said alkylation reactor to produce an alkylate stream and a spent acid stream, and
ii) de-gassing the spent acid stream thereby producing a gaseous sulfur containing stream.

A particularly preferred embodiment of the invention provides an alkylation process comprising the steps of:

a) reacting olefins and isobutane in the presence of a sulfuric acid catalyst in an alkylation reactor to produce an alkylate stream, a spent acid stream, and sulfur contaminants;

b) separating said alkylate stream into a vapor stream and a liquid effluent stream, said liquid effluent stream comprising hydrocarbons and said sulfur contaminants;

c) compressing and condensing said vapor stream to produce a depropanizer feed stream, said depropanizer feed stream comprising hydrocarbons and said sulfur contaminants;

d) degassing said spent acid stream to produce a degassed spent acid stream and an acid vapor stream, said acid vapor stream comprising hydrocarbons and said sulfur contaminants; and

e) an extraction step comprising:
i) providing an extraction stream chosen from at least one stream from the group consisting of said liquid effluent stream, said depropanizer stream, acid vapor stream, and mixtures thereof; and
ii) extracting an amount of said sulfur contaminants by contacting the extraction stream with ammonia or ammonium hydroxide thereby producing a treated stream and an ammonium salt solution comprising an extracted amount of an ammonium salt derived from said sulfur contaminants.

The preferred alkylation process can further comprise:

f) incinerating said ammonium salt solution in a furnace, preferably a spent acid regeneration furnace, to produce a gaseous mixture comprising N₂, NOₓ, SO₂, SO₃;

g) contacting said gaseous mixture with oxygen in the presence of an oxidation catalyst, preferably vanadium oxide, to convert SO₂ to SO₃; and

h) contacting the gaseous mixture with water to form sulfuric acid from the SO₃ therein.

The preferred alkylation process can further comprise the step of replenishing said sulfuric acid catalyst in step a) with the sulfuric acid made in step h).

Alternatively, step b) can comprise the steps of:
i) separating said alkylate stream into a vapor stream and a liquid hydrocarbon stream;
ii) contacting the liquid hydrocarbon stream to produce an acidified hydrocarbon stream; and
(iii) separating the acidified hydrocarbon stream into an ester phase and a liquid effluent stream, said liquid effluent stream comprising hydrocarbons and sulfur contaminants.

Alternatively, the extraction step e) can comprise the steps of:
i) providing a first extraction stream chosen from one or two streams from the group consisting of said liquid effluent stream, said depropanizer stream, said acid vapor stream, and mixtures thereof thereby defining one or more unchosen streams remaining in said group, preferably wherein said depropanizer stream and said acid vapor stream are both chosen as the first extraction stream;
ii) providing a second extraction stream chosen from one of more of said unchosen streams; and
(iii) extracting a first amount of sulfur contaminants by contacting the first extraction stream with ammonia or ammonium hydroxide thereby producing a first treated stream and a first ammonium salt solution comprising an extracted amount of an ammonium salt derived from said first amount of sulfur contaminants; and optionally
iv) extracting a second amount of sulfur contaminants by contacting the second extraction stream with said first ammonium salt solution thereby producing a second treated stream and a second ammonium salt solution comprising an extracted ammonium salt derived from said second amount of sulfur contaminants.

As another alternative, the extraction step e) can comprise the steps of:
i) providing a first extraction stream chosen from one of the group consisting of said liquid effluent stream, said depropanizer stream, and acid vapor stream thereby defining two unchosen streams remaining in said group, preferably wherein said acid vapor is chosen as the first extraction stream;
ii) providing a second extraction stream chosen from one of said two unchosen streams thereby defining one unchosen stream remaining in said group and providing a third extraction stream therefrom, preferably wherein said depropanizer stream is chosen as the second extraction stream;
(iii) extracting a first amount of sulfur contaminants by contacting the first extraction stream with ammonia or ammonium hydroxide thereby producing a first treated stream and a first ammonium salt solution comprising an extracted ammonium salt derived from said first amount of sulfur contaminants;
iv) extracting a second amount of sulfur contaminants by contacting the second extraction stream with said first ammonium salt solution thereby producing a second treated stream and a second ammonium salt solution comprising an extracted ammonium salt derived from said second amount of sulfur contaminants; and
v) extracting an third amount of sulfur contaminants by contacting the said third extraction stream with said second ammonium salt solution thereby producing a third treated stream and a third ammonium salt solution comprising an extracted ammonium salt derived from said third amount of sulfur contaminants.

After the extraction step e), the alkylation process preferably further comprises:
f) incinerating said second ammonium salt solution or third ammonium salt solution in a furnace to produce a gaseous mixture comprising N₂, NOₓ, SO₂, SO₃;
g) contacting said gaseous mixture with oxygen in the presence of an oxidation catalyst to convert SO₂ to SO₃; and
h) contacting the gaseous mixture with water to form sulfuric acid from the SO₃ therein; and preferably replenishing said sulfuric acid catalyst in step a) with the sulfuric acid made in step h).

### DETAILED DESCRIPTION

The present invention provides an improved sulfuric acid catalyzed alkylation process wherein the sulfur content of one or more sulfur containing streams in the alkylation process is recovered by an extraction agent. The extraction agent of the invention comprises ammonia or ammonium hydroxide. Advantageously, the recovered sulfur content is converted to sulfuric acid, such as in a spent acid regeneration (SAR) plant, and recycled to replenish decomposed catalyst in the alkylation process.

A sulfuric acid catalyzed alkylation process typically comprises feeding sulfuric acid, olefin and isobutane into an alkylation reactor under conditions to produce a product comprising a spent acid stream (aqueous phase) and an alkylate stream (organic phase). The acid stream is separated (e.g., in an acid settler) from the alkylate stream, which becomes the reactor effluent.

The alkylate stream (reactor effluent) may be fed to a refrigeration tube bundle or other cooling means to remove heat of reaction. After cooling, part of the alkylate stream vaporizes, forming a two-phase product: a vapor stream (gas phase), and a liquid effluent stream (liquid phase). The liquid effluent stream is also called net effluent.

The liquid effluent stream comprises alkylated products and sulfuric acid. Typically, net effluent comprises about 40-60% isobutane, about 24-35% C₅ and higher hydrocarbons, about 8-28% n-butane, about 1-4% propane, up to about 300 mg/kg sulfuric acid, and traces of alkyl sulfates.

The liquid effluent stream is typically contacted with sulfuric acid to produce an acidified effluent. The acidified effluent is separated into a first hydrocarbon stream and an ester stream. These streams are separated, e.g., by gravity.

The vapor stream comprises propane, sulfur dioxide (resulting from the decomposition of sulfuric acid catalyst) and other volatiles. More particularly, the vapor stream typically comprises about 70-85% isobutane, about 2-20% n-butane, about 5-15% propane, about 2-6% C₅ and higher hydrocarbons, and up to 300 mg/kg sulfur dioxide.

The vapor stream is typically compressed and condensed. A portion of the condensed vapor stream may be recycled directly back to the alkylation process for use as refrigerant. The remaining portion of the compressed and condensed vapor component provides a depropanizer feed stream.

The spent acid stream, which comprises spent sulfuric acid from the alkylation process, is typically treated in a conventional blow-down system where it is de-gassed to provide an acid vapor stream comprising hydrocarbons and a de-gassed spent acid stream. The de-gassed spent acid, after blow-down, is typically about 90 to about 85% sulfuric acid. The de-gassed spent acid also comprises SOₓ (sulfur dioxide and sulfur trioxide), sulfuric acid-olefin adducts (termed alkyl sulfates or “esters”) and dissolved hydrocarbons. SOₓ and sulfuric acid-olefin adducts are derived from sulfuric acid catalyst. The de-gassed spent acid is typically fed to a furnace in a spent acid regeneration (SAR) plant, where it is incinerated (burned) “as is”—that is, the de-gassed spent acid is not neutralized prior to incineration.

The acid vapor stream is typically treated in a blow-down system. The acid vapor typically comprises about 50-70% isobutane, about 5-15% n-butane, about 5-15% propane, about 5-15% C₅ and higher hydrocarbons and up to about 300 mg/kg sulfur dioxide (generated from the decomposition of sulfuric acid catalyst).

As exemplified above, a typical alkylation process produces multiple streams having sulfur contaminants. Through the use of the extraction process of the invention, sulfur value can be obtained from these contaminated streams by contacting one or more of these streams and mixtures thereof with the extraction agent of the invention to produce a corresponding treated stream and ammonium salt solution, wherein the ammonium salt solution comprises an extracted amount of an ammonium salt derived from the sulfur contaminants.

The present invention's use of ammonia or ammonium hydroxide as an extraction agent results in several advantages over the conventional use of sodium hydroxide as an extraction agent; the advantages include the creation of an ammonium salt solution having: 1) comparatively higher concentration of extracted decomposition products, making incineration economically feasible; and 2) the absence of sodium, thereby avoiding the production of sodium ash during incineration. Conversely, the use of sodium hydroxide as an extraction agent results in several disadvantages including the creation of a caustic solution having 1) comparatively lower concentration of extracted sulfur contaminants, making incineration economically prohibitive; and 2) the presence of sodium, undesirably causing the production of sodium ash during incineration.

The ammonium salt solution generated by the invention can be incinerated in a furnace (preferably a spent acid regeneration furnace) in the presence of an oxygen containing gas under conditions causing the disassociation of the ammonium salt solution into sulfur dioxide, sulfur trioxide, nitrogen, and traces of nitrogen oxides. Thus, a gaseous mixture comprising N₂, NOₓ, SO₂ and SO₃ is produced. Herein the term “oxygen-containing gas” means any gas containing oxygen preferably air or oxygen-enriched air. During the incineration and thermal disassociation procedures of the present invention, temperatures are preferably kept below about 1100° C. to minimize NOₓ formation. Typically, incineration and thermal disassociation procedures of the present invention involve oxidation with an oxygen containing gas and typically utilize a slight stoichiometric excess of oxygen, for instance 2-3 volume % excess oxygen on a dry basis. Any convenient pressure may be used; processes can be operated slightly below ambient pressure to prevent leakage into the working environment.

Preferably, the ammonium salt solution is incinerated in the furnace of a spent acid regeneration plant where the ammonia content of the ammonium salt is converted principally to elemental nitrogen, and is vented to the atmosphere after scrubbing to remove any nitrogen oxides, NOₓ. By “incinerate” it is meant herein to contact a stream (liquid or vapor) with an oxygen-containing gas. The term “NOₓ” is used herein to describe the mixed nitrogen oxides formed during combustion processes. Nitrogen oxides formed during the incineration of the ammonium salt solution can be scrubbed using a nitrogen oxide (NOₓ) scrubber for a furnace exhaust attached to a spent acid regeneration plant. The NOₓ produced represents a minor increase for the existing NOₓ scrubber load in typical alkylation process plants.

The gaseous mixture obtained by incineration of an ammonium salt solution can then be contacted with oxygen in the presence of an oxidation catalyst, preferably vanadium oxide, to convert SO₂ to SO₃. The resulting oxidized gaseous mixture can be contacted with water to form sulfuric acid from the SO₃ therein. This reformulated sulfuric acid can then be used as catalyst replenishment.

In particular, the extraction process of the invention can be applied to one or more of three contaminated streams (and mixtures thereof) produced in a typical sulfuric acid catalyzed alkylation process: 1) a liquid effluent stream, 2) a depropanizer stream, and 3) an acid vapor stream.

In one embodiment of the invention, a liquid effluent stream and a depropanizer feed stream are contacted with ammonia or ammonium hydroxide to produce an ammonium salt solution and a treated stream. The ammonium salt solution and treated stream are separated. The ammonium salt solution can be further processed as described above.

In another embodiment of the invention, an acid vapor stream is contacted with ammonia or ammonium hydroxide, preferably in a blow-down scrubber, to produce an ammonium salt solution and a treated vapor stream. The treated vapor stream, which comprises hydrocarbons, can be fed to a refinery flare according to standard procedures known to those skilled in the art.

In a particularly preferred embodiment of this invention, each of: 1) a liquid effluent stream, 2) a depropanizer stream, and 3) an acid vapor stream is subjected to the extraction process of the invention. In a still more preferred embodiment, the ammonia or ammonium hydroxide is used in series to treat all three of these streams wherein an excess of ammonia or ammonium hydroxide is contacted with an acid vapor stream to produce a first ammonium salt solution, which comprises ammonia or ammonium hydroxide. The first ammonium salt solution, is then contacted with a depropanizer feed stream to produce a second ammonium salt solution, which comprises ammonia or ammonium hydroxide. The second ammonium salt solution is then contacted with the liquid effluent stream to produce a third ammonium salt solution. The third ammonium salt solution is then incinerated in accordance with the invention described herein. As an alternative to this preferred embodiment, the ammonium salt solutions are produced in parallel.

As already described, the present invention utilizes an extraction agent comprising ammonia or ammonium hydroxide wherein the extraction agent is contacted with any hydrocarbon containing alkylation process stream to extract sulfur contaminants such as sulfur dioxide and alkyl sulfates. Because of the attendant disadvantages of sodium hydroxide's use, a preferred embodiment of the invention excludes the use of an extraction agent comprising more than 0.0004 wt. % sodium hydroxide. An even more preferred us an embodiment excludes the use of an extraction agent comprising any amount of sodium hydroxide. In another preferred embodiment, the extraction agent comprises ammonia or ammonium hydroxide and is substantially free (more preferably completely free except for impurities) of any other ingredient except water. In this regard the term “substantially free” means that the extraction agent comprises no other ingredient excess of 0.0004 wt. % except ammonia, ammonium hydroxide or water.

In a particularly preferred embodiment, the extraction agent comprises from 100 mole % ammonia (anhydrous ammonia) to about 5 mole % ammonia in water, more preferably 50 mole % to about 15 mole % ammonia in water, and even more preferably 35 mole % to about 25 mole % ammonia in water. Selection of the optimum ammonia concentration for a specific stream in a specific plant is determined on the basis of economics, including fuel costs and waste stream disposal costs.

When used as an extraction agent, the amount of ammonia contacted with an alkylation process stream is preferably in slight excess over the stoichiometric amount needed to convert all of sulfur contaminants in the stream to their ammonium salts. The time for complete conversion and neutralization typically depends on the distribution of the sulfur species. Sulfur oxides and particularly sulfur acids react and transfer the ammonium salt to an aqueous phase more quickly than sulfur esters (alkyl sulfates). Vigorous mixing can be used to maximize contact area between the aqueous phase (extraction agent) and the organic phase (alkylation process stream) accelerates the conversion of sulfur esters to the ammonium salts. Contact times with agitation of up to about one hour may be needed. Complete neutralization can be shown in a sample containing excess ammonia by a pH that is stable to continued agitation.

Initially, the ammonium salt solutions resulting from contacting the extraction agent with an alkylation process stream are typically warm (e.g., about 120° F., 50° C.). As the initial temperature cools, ammonium salt precipitation may occur. This precipitation is preferably prevented (to avoid loss of sulfur value prior to incinerating the ammonium salt solution) by diluting the warm ammonium salt solution. The amount of dilution required can be determined by performing a total dissolved solids (TDS) analysis. The preferred ammonium salt solution contains the minimum practical water content such that salt precipitation does not occur.

As already described, the present involves the use of ammonia or ammonium hydroxide to extract sulfur contaminants from a hydrocarbon containing alkylation process stream. The sulfur contaminants typically comprise: sulfur dioxide, sulfur trioxide, left over sulfuric acid, alkyl sulfates, and mixtures thereof. The ammonia or ammonium hydroxide extraction agent of the invention reacts with the sulfur contaminants to from a solubilized corresponding ammonium salt derivative. Examples of these reactions which form solubilized ammonium salts are provided below.

Illustrative reactions for ammonium hydroxide with sulfur dioxide are:

SO₂+2 NH₄OH→(NH₄)₂SO₃+H₂O

SO₂+NH₄OH→(NH₄)HSO₃

The corresponding illustrative reactions for ammonium hydroxide with sulfur trioxide are:

SO₃+2 NH₄OH→(NH₄)₂SO₄+H₂O

SO₃+NH₄OH→(NH₄)HSO₄

The corresponding illustrative reactions for ammonium hydroxide with sulfuric acid are:

H₂SO₄+2 NH₄OH→(NH₄)₂SO₄+2 H₂O

H₂SO₄+NH₄OH→(NH₄)HSO₄+H₂O

Illustrative reactions for ammonia with sulfur dioxide are:

SO₂+2 NH₃+H₂O→(NH₄)₂SO₃

SO₂+NH₃+H₂O→(NH₄)HSO₃

The corresponding illustrative reactions for ammonia with sulfur trioxide are:

SO₃+2 NH₃+H₂O→(NH₄)₂SO₄

SO₃+NH₃+H₂O→(NH₄)HSO₄

The corresponding illustrative reactions for ammonia with sulfuric acid are:

H₂SO₄+2 NH₃→(NH₄)₂SO₄

H₂SO₄+NH₃→(NH₄)HSO₄

With alkyl sulfates, such as n-butyl hydrogen sulfate, and ammonium hydroxide, the illustrative reaction is:

(C₄H₉)HSO₄+2 (NH₄OH)→C₄H₉OH+(NH₄)₂SO₄+H₂O

With ammonia the corresponding illustrative reaction is:

(C₄H₉)HSO₄+2 NH₃+H₂O→C₄H₉OH+(NH₄)₂SO₄

The products, (NH₄)HSO₃, (NH₄)HSO₄, (NH₄)₂SO₃, (NH₄)₂SO₄ shown above may all be components of the ammonium salt solution which can be introduced into the furnace of a spent acid regeneration plant to recover sulfuric acid. Under reaction conditions, the majority of the ammonia or ammonium component of the salt is converted to N₂. In addition, the alkanol, C₄H₉OH, produced from reaction of alkyl sulfate with ammonia or ammonium hydroxide remains a component of the ammonium salt solution and is incinerated.

Thus, significant advantages of the process of the present invention are obtained, including: a) the elimination of the sodium sulfate wastewater stream (typically created by conventional use of sodium hydroxide) so that incineration is economically desirable, and b) the recovery of the sulfur content as sulfuric acid, which can be re-used for catalyst replenishment.

In another embodiment of the present invention, ammonium sulfate can be crystallized from the ammonium salt solution thereby providing economic value such as use of the ammonium sulfate in fertilizer.

### EXAMPLES

The weight percent sulfur content in all of the examples below was measured by ultraviolet fluorescence in accordance with ASTM D5453.

The extraction process in all of the examples below was performed placing about 100 mL of an extraction agent and about 300 mL of a pre-treatment effluent in a three-impeller shaft continuously stirred tank reactor. The reactor temperature was maintained at a certain extraction temperature and continuously stirred at about 1,400 rpm for about 30 minutes. The reactor contents were then transferred into a beaker and the excess isobutane was allowed to vaporize until the sample had essentially no vapor pressure at room temperature thereby obtaining a treated effluent.

The hydrocarbon effluent used as pre-treatment effluent in all the examples below 1 was made by feeding sulfuric acid catalyst, isobutane, and olefins in a reactor under conditions provided in the table below to provide a hydrocarbon effluent which was collected over a nine hour period, de-pressurized, and measured to have a total sulfur content of 0.043 wt. %.

| Reaction Conditions | Target Setting |
|---|---|
| Reaction Temperature, ° F. | 65 |
| Olefin Space Velocity, Hr⁻¹ | 0.25 |
| Average Acid Strength, Wt % | 88.5 |
| Molar Isobutane to Olefin Ratio | 4 |
| Molar Diluent to Isobutane Ratio | 0.12 |
| Molar C₃/nC₄ ratio | 0.2 |

### Comparative Example A

An extraction was performed using 12 wt. % aqueous sodium hydroxide (NaOH) solution as the extraction agent and 120° F. as the extraction temperature. The total sulfur content of the treated effluent was 0.0039 wt. % representing an 8% removal of sulfur.

### Example 2

Comparative Example B was repeated but instead of using sodium hydroxide, an ammonium hydroxide solution (90 mol % ammonia in water) was used as the extraction agent. The total sulfur content of the treated effluent was 0.0020 wt. % representing a 53% removal of sulfur.

### Example 3

Comparative Example B was repeated but instead of using sodium hydroxide, an ammonium hydroxide solution (70 mol % ammonia in water) was used as the extraction agent. The total sulfur content of the treated effluent was 0.0025 wt. % representing a 41% removal of sulfur.

### Example 4

Comparative Example B was repeated but instead of using sodium hydroxide, an ammonium hydroxide solution (30 mol % ammonia in water) was used as the extraction agent. The total sulfur content of the treated effluent was 0.0021 wt. % representing a 51% removal of sulfur.

### Example 5

Example 4 was repeated except 160° F. was used as the reaction temperature. The total sulfur content of the treated effluent was 0.0015 wt. % representing a 65% removal of sulfur.

TABLE 1

| | | Wt. % | Wt. % | | |
|---|---|---|---|---|---|
| | Extraction | ammonia in | sulfur | Wt. % sulfur | |
| | temperature | extraction | before | after | % sulfur |
| Ex# | (° F.) | agent | extraction | extraction | removal |
| A | 120 | 0 | 0.0043 | 0.0039 | 8 |
| 1 | 120 | 90 mol % | 0.0043 | 0.0020 | 53 |
| 2 | 120 | 70 mol % | 0.0043 | 0.0025 | 41 |
| 3 | 120 | 30 mol % | 0.0043 | 0.0021 | 51 |
| 4 | 160 | 30 mol % | 0.0043 | 0.0015 | 65 |

The results from all the examples are summarized in Table 1 above. Comparing Examples 1-4 to Example B, demonstrates that ammonium hydroxide extracts significantly more sulfur from comparable pre-treatment effluent.

What is claimed is:

1. An alkylation process comprising the steps of:
a) generating at least one sulfur containing stream by reacting olefins and isobutane in the presence of a sulfuric acid catalyst in an alkylation reactor to produce said sulfur containing stream which comprises hydrocarbons and sulfur contaminants;
b) extracting an amount of the sulfur contaminants from said sulfur containing stream by contacting said sulfur containing stream with ammonia or ammonium hydroxide to produce a treated stream and an ammonium salt solution comprising an extracted amount of an ammonium salt derived from said sulfur contaminants; and
c) separating said ammonium salt solution from the treated stream.

2. The process of claim 1 further comprising the steps of:
d) incinerating said ammonium salt solution in a furnace to produce a gaseous mixture comprising N₂, NO_{X}, SO₂, SO₃;
e) contacting said gaseous mixture with oxygen in the presence of an oxidation catalyst to convert SO₂ to SO₃; and
f) contacting said gaseous mixture with water to form sulfuric acid from the SO₃ therein.

3. The process of claim 2 wherein the oxidation catalyst is vanadium oxide.

4. The process of claim 2 further comprising the step of replenishing said sulfuric acid catalyst in step a) with the sulfuric acid made in step f).

5. The process of claim 1 wherein at least one sulfur containing stream is liquid made by a method comprising the steps of:
i) reacting said olefins and said isobutane in the presence of said sulfuric acid catalyst in said alkylation reactor to produce an alkylate stream and a spent acid stream;
ii) separating the alkylate stream into a vapor stream and a liquid effluent stream; and
iii) compressing and condensing the vapor stream thereby forming the sulfur containing stream.

6. The process of claim 1 wherein at least one sulfur containing stream is liquid made by a method comprising the steps of:
i) reacting said olefins and said isobutane in the presence of said sulfuric acid catalyst in said alkylation reactor to produce an alkylate stream and a spent acid stream;
ii) separating the alkylate stream into a vapor stream and a liquid effluent stream;
iii) optionally contacting the liquid effluent stream with an acid; and
iv) obtaining the sulfur containing stream from the liquid component.

7. The process of claim 1 wherein at least one sulfur containing stream is gas made by a method comprising the steps of:
i) reacting olefins and isobutane in the presence of said sulfuric acid catalyst in said alkylation reactor to produce an alkylate stream and a spent acid stream, and
ii) de-gassing the spent acid stream thereby producing a gaseous sulfur containing stream.

8. An alkylation process comprising the steps of:
a) reacting olefins and isobutane in the presence of a sulfuric acid catalyst in an alkylation reactor to produce an alkylate stream, a spent acid stream, and sulfur contaminants;
b) separating said alkylate stream into a vapor stream and a liquid effluent stream, said liquid effluent stream comprising hydrocarbons and said sulfur contaminants;
c) compressing and condensing said vapor stream to produce a depropanizer feed stream, said depropanizer feed stream comprising hydrocarbons and said sulfur contaminants;
d) de-gassing said spent acid stream to produce a degassed spent acid stream and an acid vapor stream, said acid vapor stream comprising hydrocarbons and said sulfur contaminants; and
e) an extraction step comprising:
i) providing an extraction stream chosen from at least one stream from the group consisting of said liquid effluent stream, said depropanizer stream, acid vapor stream, and mixtures thereof; and
ii) extracting an amount of said sulfur contaminants by contacting the extraction stream with ammonia or ammonium hydroxide thereby producing a treated stream and an ammonium salt solution comprising an extracted amount of an ammonium salt derived from said sulfur contaminants.

9. The process of claim 8 further comprising:
f) incinerating said ammonium salt solution in a furnace to produce a gaseous mixture comprising N₂, NOₓ, SO₂, SO₃;
g) contacting said gaseous mixture with oxygen in the presence of an oxidation catalyst to convert SO₂ to SO₃; and
h) contacting the gaseous mixture with water to form sulfuric acid from the SO₃ therein.

10. The process of claim 9 wherein the oxidation catalyst is vanadium oxide.

11. The process of claim 9 further comprising the step of replenishing said sulfuric acid catalyst in step a) with the sulfuric acid made in step h).

12. The process of claim 8 wherein step b) alternatively comprises the steps of:
i) separating said alkylate stream into a vapor stream and a liquid hydrocarbon stream;
ii) contacting the liquid hydrocarbon stream to produce an acidified hydrocarbon stream; and
iii) separating the acidified hydrocarbon stream into an ester phase and a liquid effluent stream, said liquid effluent stream comprising hydrocarbons and sulfur contaminants.

13. The process of claim 8 wherein the extraction step e) alternatively comprises the steps of:
i) providing a first extraction stream chosen from one or more streams from the group consisting of said liquid effluent stream, said depropanizer stream, said acid vapor stream, and combinations thereof thereby defining one or more unchosen streams remaining in said group;
ii) providing a second extraction stream chosen from one of more of said unchosen streams; and
iii) extracting a first amount of sulfur contaminants by contacting the first extraction stream with ammonia or ammonium hydroxide thereby producing a first treated stream and a first ammonium salt solution comprising an extracted ammonium salt derived from said first amount of sulfur contaminants.

14. The process of claim 13 wherein the extraction step e) further comprises the step of:
iv) extracting a second amount of sulfur contaminants by contacting the second extraction stream with said first ammonium salt solution thereby producing a second treated stream and a second ammonium salt solution comprising an extracted ammonium salt derived from said second amount of sulfur contaminants.

15. The process of claim 13 wherein said depropanizer stream and said acid vapor stream are both chosen as the first extraction stream.

16. The process of claim 7 wherein the extraction step e) alternatively comprises the steps of:
i) providing a first extraction stream chosen from one of the group consisting of said liquid effluent stream, said depropanizer stream, and acid vapor stream thereby defining two unchosen streams remaining in said group;
ii) providing a second extraction stream chosen from one of said two unchosen streams thereby defining one unchosen stream remaining in said group and providing a third extraction stream therefrom;
iii) extracting a first amount of sulfur contaminants by contacting the first extraction stream with ammonia or ammonium hydroxide thereby producing a first treated stream and a first ammonium salt solution comprising an extracted ammonium salt derived from said first amount of sulfur contaminants;
iv) extracting a second amount of sulfur contaminants by contacting the second extraction stream with said first ammonium salt solution thereby producing a second treated stream and a second ammonium salt solution comprising an extracted ammonium salt derived from said second amount of sulfur contaminants.
v) extracting an third amount of sulfur contaminants by contacting the said third extraction stream with said second ammonium salt solution thereby producing a third treated stream and a third ammonium salt solution comprising an extracted ammonium salt derived from said third amount of sulfur contaminants.

17. The process of claim 13 wherein said acid vapor is chosen as the first extraction stream.

18. The process of claim 13 wherein said depropanizer stream is chosen as the second extraction stream.

19. The process of claim 13 further comprising the steps of:
f) incinerating said second ammonium salt solution or third ammonium salt solution in a furnace to produce a gaseous mixture comprising N₂, NOₓ, SO₂, SO₃;
g) contacting said gaseous mixture with oxygen in the presence of an oxidation catalyst to convert SO₂ to SO₃; and
h) contacting the gaseous mixture with water to form sulfuric acid from the SO₃ therein.

20. The process of claim 19 further comprising the step of replenishing said sulfuric acid catalyst in step a) with the sulfuric acid made in step h).
